(19) United States
(12) Patent Application Publication
(10) Pub. No.: US 2023/0404482 A1
(43) Pub. Date: Dec. 21, 2023

(54) ELECTROMAGNETICALLY SHIELDED REMOVABLE PLUG FOR USE IN AN ELECTROMAGNETIC FIELD CHANNEL

(71) Applicants: Dan Miulli, Riverside, CA (US); James Garvin Wiginton, IV, Riverside, CA (US); James Brazdzionis, Redlands, CA (US); Paras Savla, Riverside, CA (US)

(72) Inventors: Dan Miulli, Riverside, CA (US); James Garvin Wiginton, IV, Riverside, CA (US); James Brazdzionis, Redlands, CA (US); Paras Savla, Riverside, CA (US)

(21) Appl. No.: 18/321,073

(22) Filed: May 22, 2023

Related U.S. Application Data
(63) Continuation of application No. 18/149,980, filed on Jan. 4, 2023.
Application No. 18/149,980 is a continuation of application No. 18/172,676, filed on Feb. 22, 2023.
(60) Provisional application No. 63/365,048, filed on May 20, 2022.
(60) Provisional application No. 63/365,051, filed on May 20, 2022.
(60) Provisional application No. 63/365,049, filed on May 20, 2022.

(51) Int. Cl. A61B 5/00; H05K 9/00; A61B 5/291

(52) U.S. Cl. CPC A61B 5/6803; H05K 9/0049; A61B 5/291; A61B 2562/227; A61B 2562/0209; A61B 2562/182; H05K 9/006

(57) ABSTRACT

A plug for an electromagnetic field channel. The plug may have a tubular body capped at one end. The body may be composed of a plurality of coaxial layers, including electromagnetically inert outermost and innermost layers. A MU-metallic second layer may be wrapped by the outermost electromagnetically inert layer, and a copper third layer may be wrapped by the innermost electromagnetically inert layer. An air gap may be disposed between the second and third layers.

### CROSS-REFERENCE TO RELATED APPLICATION

This application claims the benefit of priority of U.S. provisional applications—No. 63/365,048, filed May 20, 2022; No. 63/365,051, filed May 20, 2022; and No. 63/365,049 filed 20 May 2022, and claims the benefit of priority of U.S. non-provisional patent applications—Ser. No. 18/149,980, filed 4 Jan. 2023; and Ser. No. 18/172,676, filed 22 Feb. 2023, as a continuation thereof, the contents of each are herein incorporated by reference.

### BACKGROUND OF THE INVENTION

The present invention relates to an electromagnetic field channel and, more particularly, to an electromagnetically shielded removable plug for use therefor.

An electromagnetic channel is a channel that allows for the placement of a housed sensor for measuring a target electromagnetic field (EMF) therein.

From time to time, there could be multiple channels spaced apart and/or provided on one omnibus device of a test subject, say on a helmet of a subject wearer/user. In this scenario, each channel offers its own determination of different target EMF at different locations along the subject wearer/user of the omnibus device. When a channel is not in use but adjacent to a channel of interest being used for sensing, electromagnetic energy can leak, escape, or enter through and/or from the empty channel, and thereby diluting the adjacent subject channel's ability to appropriately measure the electromagnetic field.

Currently, there does not exist a plug that achieves this result in this manner. Currently these specific electromagnetic field channels do not have a plug, thereby allowing external signal to enter the channel and helmet reducing shielding capabilities. The electromagnetic field of a subject is also allowed to escape, or an external electromagnetic field can enter through empty channels if a plug is not in use. The presence of empty channels allows for a shield to be used in various configurations and the plug allows for temporary shielding from any empty channels not currently in use.

Currently there are no systems on the market designed to temporarily plug unused channels of an electromagnetic shield having a plurality of electromagnetic channels, which would allow for such a shield to be modular for usage in healthcare technologies and research. Specifically, there are also no plugs designed for electromagnetic field channels that interface with such a shield and channels.

As can be seen, there is a need for a plug that can electromagnetically shield an electromagnetic field channel for a modular shielding device, wherein the plug does not impact the level of shielding.

### SUMMARY OF THE INVENTION

The present invention embodies a plug for an electromagnetic channel, wherein the plug provides Mu-metal® and copper layers for attenuation of magnetic fields and electrical fields, respectively, stopping passage of an electromagnetic field through an otherwise empty electromagnetic field channel.

The plug embodies two stacks of Mu-metal® and copper layers in a spaced apart relation within the tubular body of the plug. And air gaps between the two stacks provide critical insulation. Magnetic fields and electrical fields are reflected by the Mu-metal and copper layers, respectively. An external plastic layer on each stack provides structural support and further insulation from both external and internal magnetic fields.

An embodiment of the present invention comprises a plug provides an area of shielding in a hollow portion of an electromagnetic channel. Multi-layered nickel-iron soft ferromagnetic alloy, such as Mu-metal®, copper mesh, and plastic provide substrate in the exact dimensions to fill the void inside the electromagnetic channel when a sensor is not present. This is achieved with a specific configuration of layers to achieve adequate shielding.

A top plastic cap prevents movement of the plug further into the EMF channel thereby prohibiting it from being easily removed.

This device may function as a plug to go inside an electromagnetic field channel or as a cap for gaps in multilayer Mu-metal® and interlaced copper mesh shielding, when the gap is not being utilized for sensing.

As mentioned above, sensors may be utilized to measure electromagnetic fields contained within shielded materials or shielded containers. In order to place the sensors within the shielded container there must be a gap for the sensor to pass through. If multiple gaps/channels are provided so that sensors can be placed in a variety of configurations, a cap or plug is required to plug the space not being utilized to prevent the corruption of a nearby, desired signal within a target channel. This plug needs to functionally act as the shield even if the space is plugged, else an electromagnetic field from the external environment may enter the shielded container and create aberrant readings.

The plug embodied in the present invention is specifically designed to be placed in the end of an electromagnetic field channel, into a channel in a specifically designed device. This plug may also be manufactured in a variety of shapes or sizes to fill gaps in multilayer Mu-metal® and interlaced copper mesh shielding not being utilized for sensing. The Mu-metal® may be constructed of a nickel-iron alloy, soft ferromagnetic alloy, and or the like, and the copper layer may be interlaced copper mesh.

The plug of the present invention is the first system designed to plug the holes within electromagnetic field channels designed with copper mesh and Mu-metal® to improve shielding technologies. This allows for a modular approach to move EMF sensors to other regions to measure differing areas of interest with sensors all while preserving the shielding integrity.

The Mu-metal® layers provide attenuation for the magnetic field coming into an empty electromagnetic field channel. The copper layers provide attenuation for the electrical field coming into the channel. The air gap provides further insulation. The outer Mu-metal® tubular layer provides structural support and further insulation from both external and internal magnetic field. The top plastic cap prevents movement of the plug further into the EMF channel thereby prohibiting it from being easily removed. The internal and external plastic discs provide structural support.

In one aspect of the present invention, a plug for an electromagnetic field channel includes a tubular body; and two electromagnetic shield stacks spaced apart by an airgap, wherein each electromagnetic shield is coextensive with an inner cross-sectional area of the tubular body.

In another aspect of the present invention, a plug for an electromagnetic field channel includes the following: a tubular body of MU-metal; two electromagnetic shield stacks spaced apart by an airgap having a longitudinal length of approximately one inch in length, wherein each electromagnetic shield is coextensive with an inner cross-sectional area of the tubular body; each electromagnetic shield stack having: an outermost electromagnetically inert layer; an intermediate copper mesh layer; and an innermost MU-metal layer, wherein each layer is adhered to an adjacent layer; and a cap of electromagnetically inert attached to one end of the tubular body, wherein the cap has a perimeter greater than a perimeter of the tubular body.

These and other features, aspects and advantages of the present invention will become better understood with reference to the following drawings, description, and claims.

### BRIEF DESCRIPTION OF THE DRAWINGS

FIG. 1 is a perspective view of an exemplary embodiment of the present invention, shown in use.

FIG. 2 is an exploded perspective view of an exemplary embodiment of the present invention, shown in use.

FIG. 3 is a section view of an exemplary embodiment of the present invention, taken along line 3-3 in FIG. 2.

FIG. 4 is a similar view to FIG. 3 but showing the electromagnetic plug in an installed condition.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Currently these specific electromagnetic field channels do not have a plug, thereby allowing external signal to enter the channel and helmet reducing shielding capabilities. Specifically, an electromagnetic field of a subject channel is distorted through an extrinsic electromagnetic propagating from an empty channel if a plug is not in use. In short, the presence of empty channels allows for a shield to be used in various configurations, yet the presence of empty channels invites the risk of distortion in a target or subject channel, and so the plug allows for temporary shielding from any empty channels not currently in use.

The plug of the present invention is the first system designed to plug the holes within electromagnetic field channels designed with copper mesh and Mu-metal® to improve shielding technologies. This allows for a modular approach to move electromagnetic field (EMF) sensors to other regions to measure differing areas of interest with sensors all while preserving the shielding integrity.

The description herein repeatedly refers to Mu-metal®. It is noted that the present invention is not particularly limited to Mu-metal®. When Mu-metal® is discussed, any proper substitute may be utilized.

Broadly, an embodiment of the present invention comprises a plug provides an area of shielding in a hollow portion of an electromagnetic channel. Multi-layered nickel-iron soft ferromagnetic alloy, such as Mu-metal® and operatively associated copper mesh, along with an electromagnetically inert sheet, may be dimensioned and shaped in the exact dimensions to fill the void inside the electromagnetic channel when a sensor is not present. This is achieved with a specific spaced apart relationships and arrangement of two stacks of layers to achieve adequate shielding.

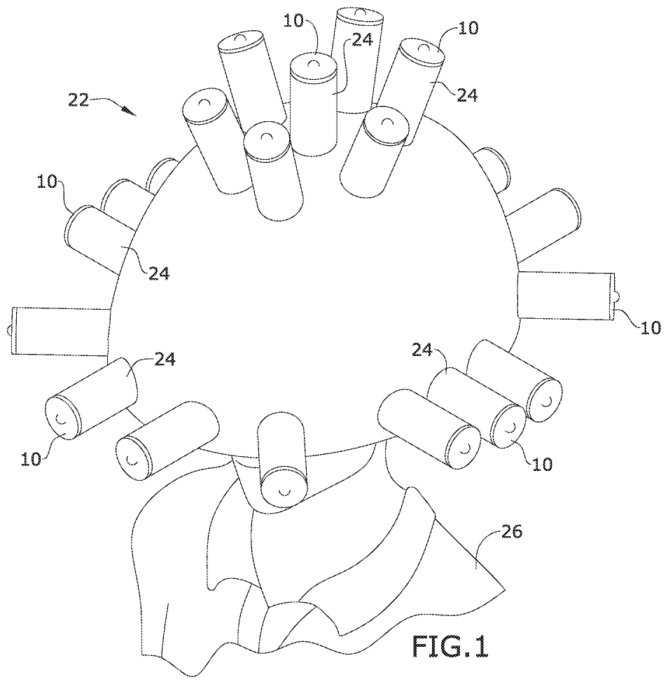

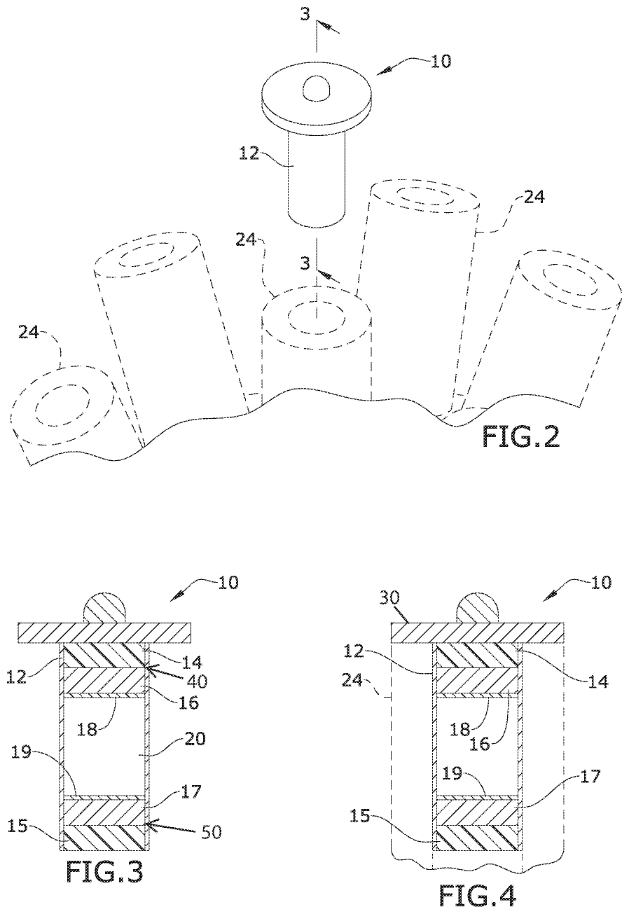

Referring now to FIGS. 1 through 4, the present invention includes an electromagnetic plug (EMFP) 10 for filling an electromagnetic field channel. The EMFP 10 includes a cap 30 from which perpendicularly extends a tubular plug body 12. Cap 30 has a diameter greater than the body 12, and thus the cap overhangs along the entire circumference of the plug body 12. The body 12 may be made or comprised of Mu-metal.

The EMFP 10 provides two stacks 40, 50 spaced apart from each other within the body 12. Each stack 40, 50 may be coextensive with an inner diameter of the tubular body 12. The first stack 40 may disposed on a first end of the body 12, adjacent the cap 30. The second stack 50 may be disposed at a second end, opposite the first end. Between the first stack 40 and the second stack 50 is an air gap 20 having a distance (along the longitudinal axis of the tubular body 12) of between three-quarters to one and one-quarter inch). The air gap 20 may have a uniform cross-section throughout its length.

For each stack 40, 50, an outermost layer 14 and 15, respectively, may be made of an electromagnetically inert material, such as but not limited to plastic. By “outermost”, reference is made to FIGS. 3 and 4, indicating further from a center of the tubular body 12. Just inward of the outermost layer 14 and 15, for each stack 40 and 50, is an intermediate copper mesh layer 16 and 17, respectively. The innermost layer for each stack 40 and 50 is a Mu-metal layer 18 and 19, respectively. Thus, the outermost layer and the innermost layer sandwich the intermediate layer. Within each stack 40, 50, the layers may be adhered together with adhesive.

Each stack 40, 50, and each respective sandwich of layers for each stack, are coextensive with an inner diameter/periphery of the tubular body 12. Also, if tubular body 12 is cylindrical, the layers will be discs; though, if the tubular body 12 is rectangular then the layers will be non-rectangular, etc.

The intermediate and innermost layers 16, 18 and 17, 19, respectively, are necessary and critical for the function of the present invention. The air gap 20 is also critical to provide the necessary buffer between the two stacks. The outermost layers may be plastic, but other materials are contemplated for the first layer if they enable or do not disenable the functions of the present invention disclosed herein. For example, if the outermost layer material is electromagnetically inert (e.g., does not modify the electromagnetic field) and provides the structure integrity, like plastic does, for at least one other layer.

The cap 30 may also may be made of electromagnetically inert material/plastic. The plastic cap 30 may be placed on top of the first stack 40 and secured thereto with adhesive. The cap 30 provides surface area for the plug 10 to not slip into the EMF channel 24 it is plugged into. The outer diameter of the body 12 is customized to perfectly fit inside an electromagnetic field channel 24 or opening within shielding.

The Mu-metal layers 18 and 19 provide attenuation of the magnetic field. The intermediate copper mesh layers 16 and 17 are outward of the Mu-metal layers 18 and 19. The copper attenuates the electric field, preventing an electromagnetic field from entering or existing the channel 24. The thickness of the copper mesh layers 16 and 17 is approximately 0.25 inches. The plastic disc thickness can be variable. The Mu-metal® layer 14 is approximately 0.014 inches thick. Between the spaced apart sandwich stacks 40 and 50 is the airgap 20. In some embodiments, the airgap 20 may comprise electromagnetically inert material.

In use, the EMF channel 24 may be tubular and dimensioned to receive a sensor configured to measure electromagnetic fields. These sensors are reliant on shielding to exclude external electromagnetic fields. The sensors can be placed within the EMF channel at a desired distance away from a target, such as the human brain. Accordingly, one or more EMF channels 10 can be connected to an omnibus shielding device 22, such as a helmet, that may contain the subject or object of interest, and that can be worn by a user 26.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the invention and that modifications may be made without departing from the spirit and scope of the present invention.

What is claimed is:

1. A plug for an electromagnetic field channel, the plug comprising:
a tubular body; and
two electromagnetic shield stacks spaced apart by an airgap, wherein each electromagnetic shield is coextensive with an inner cross-sectional area of the tubular body.

2. The plug of claim 1, wherein each electromagnetic shield stack comprises:
an outer electric field attenuator layer; and
an inner magnetic field attenuator layer.

3. The plug of claim 2, wherein each electromagnetic shield stack further comprises an outermost electromagnetically inert layer.

4. The plug of claim 2, wherein the outer electric field attenuator layer comprises a copper mesh.

5. The plug of claim 2, wherein the inner magnetic field attenuator layer comprises a MU-metal.

6. The plug of claim 2, wherein the airgap is approximately one inch in length.

7. The plug of claim 2, further comprising a cap of electromagnetically inert attached to one end of the tubular body, wherein the cap has a perimeter greater than a perimeter of the tubular body.

8. The plug of claim 2, wherein the tubular body comprises a MU-metal.

9. The plug of claim 3, wherein for each electromagnetic shield stack, each layer is adhered to an adjacent layer.

10. A plug for an electromagnetic field channel, the plug comprising:
a tubular body of MU-metal; and
two electromagnetic shield stacks spaced apart by an airgap having a longitudinal length of approximately one inch in length, wherein each electromagnetic shield is coextensive with an inner cross-sectional area of the tubular body,
each electromagnetic shield stack comprises:
an outermost electromagnetically inert layer;
an intermediate copper mesh layer; and
an innermost MU-metal layer, wherein each layer is adhered to an adjacent layer; and
a cap of electromagnetically inert attached to one end of the tubular body, wherein the cap has a perimeter greater than a perimeter of the tubular body.
